(19) United States
(12) Patent Application Publication
(10) Pub. No.: US 2015/0297592 A1
(43) Pub. Date: Oct. 22, 2015

(54) COMPOSITION CONTAINING ECOTINE OR HYDROXYECOTINE AS AN ACTIVE SUBSTANCES FOR PROMOTING THE REGENERATION OF INJURED BODY TISSUE

(71) Applicants: Andreas BILSTEIN, Bergheim (DE); Olaf CHERNER, Dortmund (DE); Georg LENTZEN, Wesel (DE)

(72) Inventors: Andreas Bilstein, Bergheim (DE); Olaf Scherner, Dortmund (DE); Georg Lentzen, Wesel (DE)

(21) Appl. No.: 14/413,061

(22) PCT Filed: Jun. 19, 2013
(86) PCT No.: PCT/EP2013/062708

§ 371 Date: Jan. 6, 2015

(30) Foreign Application Priority Data
Jul. 9, 2012 (DE) 10 2012 013 482.7

(51) Int. Cl. A61K 31/505; A61K 45/06; A61K 31/7032; C07D 239/06; C07H 15/04

(52) U.S. Cl. CPC A61K 31/505; C07D 239/06; C07H 15/04; A61K 31/7032; A61K 45/06

(57) ABSTRACT

The invention relates to a composition containing as active agent ectoine, hydroxyectoine, glucosylglycerol and/or salts, esters or amides of these compounds for promoting the regeneration of injured body tissue. The invention has special significance for the treatment of chronic wounds or ulcers.

The invention relates to a composition by means of which the regeneration of injured body tissue can be promoted.

Body tissue may suffer injuries in various ways. Tissue injuries may in particular occur through external influences, as a result of traumatic events. Tissue injuries of this nature are generally also referred to as wounds, especially where the skin or mucous membranes are concerned. Aside from tissue injuries caused by external influences, other injuries in the form of ulcers are also known that are not attributable to traumatic events.

As a rule, the regeneration of impaired body tissue in a natural way already begins shortly after the injury has occurred. In the event a wound has been inflicted the blood coagulation starts very quickly causing the damaged blood vessel to be closed off by a blood clot. In the subsequent exudation phase ichor oozes out resulting in foreign bodies and germs to be discharged from the wound. The immune system then attacks and kills bacteria.

New connective tissue develops during a subsequent proliferation phase so that the defect caused by the wound is filled out. The wound is finally closed in a regeneration phase due to overgrowing epithelial cells progressing from intact epithelial tissue existing in the area of wound margins.

Whereas natural wound healing is relatively unproblematic as a rule with wounds of not too great a size, complications may occur in the event of serious wounds inter alia as a result of an excessive formation of exudate. The same applies to various types of ulcers. Moreover, chronic wounds are to be regarded as particularly problematic if they have been caused by permanent pressure (decubitus), or a delayed wound healing as a result of diabetes mellitus. Patients suffering from the latter may even develop the so-called diabetic foot syndrome which is responsible for two-thirds of all amputations in Germany.

It is therefore the objective of the invention to provide an agent capable of assisting and promoting the regeneration of injured body tissue.

As proposed by the present invention this objective is accomplished by a composition containing as active agent ectoine, hydroxyectoine, glucosylglycerol and/or salts, esters or amides of these compounds for promoting the regeneration of injured body tissue.

Ectoine and hydroxyectoine are tetrahydropyrimidine derivatives which are synthesized under stress conditions in extremophilic, especially halophilic microorganisms. Various applications or uses have been described hitherto for ectoine and hydroxyectoine, for example as moisturizers, for the treatment of the vascular leak syndrome (VLS) (DE 10 2006 056 766 A1) or for the treatment of neurodermatitis (DE 103 30 243 A1). From publication DE 100 06 578 A1 the use of ectoine and its derivatives is known for protecting biopolymers against decomposition by degrading enzymes such as proteases, nucleases or lipases.

The systematic name of ectoine is 2-methyl-1,4,5,6-tetrahydropyrimidine-4-carboxylic acid and of hydroxyectoine 5-hydroxy-2-methyl-1,4,5,6-tetrahydropyrimidine-4-carboxylic acid.

The structure of natural L-ectoine ((S)-2-methyl-1,4,5,6-tetrahydropyrimidine-4-carboxylic acid) is illustrated below:

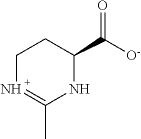

The structure of natural hydroxyectoine ((4S,5S)-5-hydroxy-2-methyl-1,4,5,6-tetrahydropyrimidine-4-carboxylic acid) is indicated hereunder:

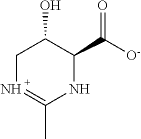

The use of the stereoisomers indicated is preferred but not obligatory, i.e. other stereoisomers or racemates may also be employed.

Glucosylglycerol, or more specific 2-O-α-D-glucosylglycerol, is a natural substance synthesized, for example, from cyanobacteria which make use of its properties for osmoprotective purposes. In this way, cyanobacteria are capable of growing in saline media with concentrations of up to 1.5 M NaCl. The molecule accumulates in high concentrations in the cytoplasm and in this way causes the existing osmotic pressure existing in such an environment due to the high salt concentration to be reduced thus protecting the cell against water losses. An example here is the cyanobacterium Synechocystis sp. PCC 6803.

Furthermore, the molecule is also synthesized by plants of genus myrothamnus. These plants are growing in humid-to-dry environments. Myrothamnus flabellifolia is a small shrub found in the southern region of Africa growing on rock slabs up to a height of 60 cm. In drought periods occurring there and lasting several months the plant survives completely unharmed and in desiccated condition. However, as soon as it rains again the plant begins to sprout within a few hours so that it is also known under the byword of “resurrection plant”. The application of glucosylglycerol as moisturizer is known in the cosmetic and dermatological fields (DE 195 40 749 A1). Moreover, as described in publication DE 10 2008 039 231 A1 glucosylglycerols are also used to augment the expression of cell protection enzymes. The structure of 2-O-α-D-glucosylglycerol is as follows:

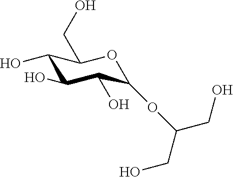

The glucosylglycerol employed is preferably the naturally occurring 2-O-α-D-glucosylglycerol which for example is accumulated by cyanobacteria of genus Synechocystis. However, comparable effects can also be expected from the β-glycosidic linkage of glucose to the glycerol molecule or from the linkage of glucose to glycerol at the 1-position. In addition to using natural occurring glucosylglycerol it is therefore conceivable as well to use also 1-O-α glucosylglycerol, 1-O-β glucosylglycerol, and 2-O-β glucosylglycerol in D and L configuration. The individual molecules (here shown in D-configuration only) are illustrated hereunder:

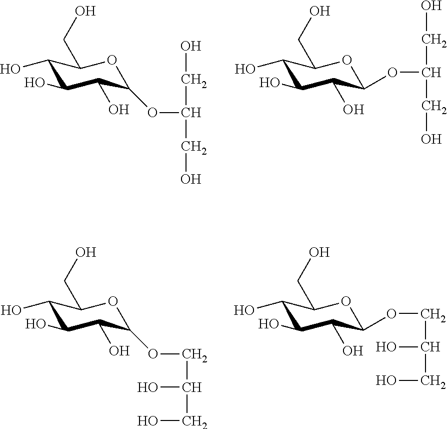

Within the scope of the present invention the body tissue the regeneration of which can be promoted by compositions containing ectoine, hydroxyectoine or glucosylglycerol may in particular be skin or mucous membranes. The injury may in particular be of traumatic nature. This means, the injury has been caused by external influences, for example by kicks, cuts, stabs, bites or the like. Such mechanically inflicted wounds may have been caused by accidents or be the result of surgical operations.

An impairment of the mucous membranes may also be brought about by mucositis the treatment thereof shall also fall within the scope of this invention relating to promoting the regeneration of injured body tissue. Mucositis can have various causes. Due to the fact that the regeneration rate of mucous membrane cells is high mucositis, for example, frequently occurs as an adverse effect of cancer treatment during chemotherapy or radiotherapy. What is more, a weakened immune system as it exists, for example, in immunocompromised patients increasingly leads to infections that may then result in an inflammation of the mucous membrane. Especially the mucous membranes of the mouth as well as those of the gastrointestinal tract may be affected.

Aside from mechanically inflicted wounds there are further tissue injuries of other nature that can also be treated with the composition proposed by the present invention. This includes, for example, thermal wounds resulting from heat, fire, scalding or frostbite, burn wounds, chemical burns or wounds stemming from ionizing radiation.

In addition to wounds also contusion/bruises can be treated with the aid of the inventive composition. Contusion causes organs or body parts to be impaired as a result of mechanical force being exerted without the skin itself being injured. Especially when in this case the blood exits damaged capillaries and ingresses into surrounding tissue hematomata are caused.

In addition to promoting the regeneration of injured body tissue in the event such injury is caused by external influences the composition as proposed by the invention is also suited for the treatment of ulcers. Ulcers may develop for various reasons, for instance as a result of circulatory disorders, tumors or infections. Examples of ulcers that can be treated with the help of the composition in accordance with the invention are ulcus cruris (“ulcerated leg”), decubitus (pressure ulcer), malum perforans (pressure ulcer on the foot), ulcus durum. ulcus molle, ulcus rodens, ulcus corneae, and others.

The composition has special significance for the treatment of chronic injuries, in particular chronic wounds or chronic ulceration. An example in this context is the diabetic foot syndrome (DFS), colloquially referred to as diabetic foot. In this connection slight injuries are initially incurred, especially of the foot or lower leg, that would normally heal without complications but due to the poor wound healing diabetes patients are prone to are frequently of long-term nature. Among other factors, the circulatory disorders diabetic patients suffer from impair the proper wound healing process. Characteristic of the diabetic foot syndrome are ulcers that may deeply progress into that part of the body, with the additional risk that germ-induced infections may occur. The number of amputations that must be performed each year due to the diabetic foot syndrome is considerable so that the provision of an effective treatment possibility is desirable and necessary.

Another frequently occurring tissue impairment is known by the term decubitus (decubital ulcer, pressure ulcer). Especially people in need of nursing care and confined to bed suffer from decubitus due to the fact that pressure is permanently exerted on certain parts of the body. In the event the pressure acting on the vessels exceeds the capillary pressure of the vessels insufficient amounts of oxygen as well as nutrients are transported to the cells finally resulting in tissue damage. Whereas pressure ulcers as a rule do not occur in healthy people because they reposition themselves regularly thus eliminating the pressure acting on endangered skin areas these reflexes are restricted or exist only to a limited degree in care-dependent persons. Decubitus may in particular occur in skin areas where bones are positioned close to the surface of the skin. There is also a danger that an open decubital ulcer allows the penetration of micro-organisms. In view of the great number of people in need of nursing care and the serious consequences the occurrence of decubitus leads to it is especially this indication that calls for beneficial treatment options.

Other injuries of body tissue the composition proposed by the invention can be applied to are hemorrhoids injuries or anal fissures which in most cases are caused by mechanical stress.

Promoting the regeneration of injured body tissue as described is also of advantage insofar as the formation of scars can be counteracted in this manner. It has been found that by applying the inventive composition the healing of wounds, especially of skin wounds, is improved so that scarring undesirable for optical reasons can be avoided.

The composition proposed by the invention shall in particular be applied onto the injured body tissue which means it shall in particular be administered locally or topically. Accordingly, the composition can be provided in the form of an ointment, cream, lotion, milk, liquid, emulsion, microemulsion, spray, suspension, paste, powder or as other solid substance.

The composition may contain customary auxiliary substances, for instance carrier agents, preservation agents, bactericides, solutizers, vitamins, stabilizers, anti-foaming substances, thickeners, colorants, surfactants, emulsifiers, moisturizing substances or the like.

Ointments, pastes, creams, and gels may contain customary carrier substances such as, for example, animal and vegetable fats, waxes, paraffins, starch, gum tragacanth, cellulose derivatives, polyethylene glycols, silicones, bentonites, silicic acid, talcum and zinc oxide or mixtures/blends of these substances.

While the compositions may contain further active agents it is, however, in particular also possible and sufficient for promoting and facilitating the body tissue regeneration to use compositions that only contain as active substances ectoine, hydroxyectoine and/or glucosylglycerol or salts, esters or amides thereof.

For example, ectoine, hydroxyectoine, glucosylglycerol or, respectively, relevant derivatives may be combined with a substance or a plurality of substances to be selected from: dexpanthenol or derivatives, arnica montana extract (arnica), capsaicin, capsicum extract, hypericum perforatum extract (St John's wort), caris diospermum halicacabum (balloon plant), hamamelis virginiana extract (witch hazel), tocopherol, allantoin, bisabolol, cocoa extract, silver, nanosilver, microsilver, amorphous silver, salts of silver, zinc, zinc oxide, calendula officinalis extract (marigold), honey and honey extracts, propolis, melilotus officinalis extract, comfrey extract (symphytum), echium vulgare extract, cumin, angelica sinensis extract, ferulic acid, hyaluronic acid, aloe vera extract, matricaria recutita (chamomile) extract, allium cepa (onion) bulb extract, achillea millefolium extract (yarrow), glycyrrhiza inflate extract (licorice), licochalcon A, silicone, urea, echinacea purpurea (purple coneflower) extract, chicoric acid.

The concentration of ectoine/hydroxyectoine, glucosylglycerol and/or respective salts, esters or amides may in particular range between 0.01 and 50% w/w, especially between 0.5 and 20% w/w, and particularly preferred between 1 and 10% w/w.

### EXAMPLE 1

### Effect of Ectoine on Wound Closure In Vitro

Plating of HaCaT cells (human skin cells) took place on collagen-I coated 24-well plates. With a complete medium (10% FCS=fetal calf serum) the cells grew together as monolayer within a period of 24 h.

After this cell layer had closed a circular injury was applied in the center using a sterile tip of a defined diameter that averaged 0.68 mm (0.363 mm²).

When the injury had been applied the complete medium was changed to a starvation medium containing only 0.5% w/w of FCS (fetal calf serum) instead of 10% w/w FCS. The following test solutions were then pipetted on the cells (per solution: n=4):

0.5% FCS+PBS (phosphate buffered saline), control solution

0.5% FCS+1 mM ectoine

0.5% FCS+10 mM ectoine

0.5% FCS+100 mM ectoine

Over a time span of 48 h the cells were incubated with the test solutions during which wound closure was documented with the aid of a digital camera. In each case photos were taken at the beginning and then at 24-h intervals. The area still found to be free in each case was measured or calculated and expressed in mm². The results can be seen from the following tables (SD: Standard deviation. MW: Mean value):

| | Area of Injury in mm² | | | | | |
|---|---|---|---|---|---|---|
| | After 0 hours | | After 24 hours | | After 48 hours | |
| | MW | SD | MW | SD | MW | SD |
| Control | 0.363 | 0.026 | 0.212 | 0.046 | 0.080 | 0.040 |
| 1 mM ectoine | 0.354 | 0.012 | 0.157 | 0.028 | 0.042 | 0.037 |
| 10 mM ectoine | 0.351 | 0.033 | 0.182 | 0.034 | 0.023 | 0.029 |
| 100 mM ectoine | 0.325 | 0.008 | 0.244 | 0.011 | 0.171 | 0.009 |

With the solutions incubated with 1 mM or 10 mM of ectoine a marked tendency towards faster closure of the circular injury inflicted on the cell layer could be ascertained in comparison with the control solution used. A delayed healing of the injured tissue was found only when applying an ectoine solution of 100 mM.

### EXAMPLE 2

### Re-Epithelialization when Incubating with Ectoine/Hydroxyectoine

After 24 hours of incubation using 0, 0.1, 0.5. 1, 2.5 and 5 mM of ectoine/hydroxyectoine in serum-free and serum-containing media defined cell areas were removed (scratch assay) from a confluent cell layer of ARPE-19 cells (adherent retinal pigment epithelial cells). The width of the applied gap was determined via microscope at previously marked places. 24 and 48 hours later in each case a photo documentation was prepared with the gap width being again determined at the respective places. 6 tests were performed for each concentration.

The highest growth rates of re-epithelialization could be observed during the first two days, with the gap width in the cell cultures treated with ectoine/hydroxyectoine found to have diminished faster. Re-epithelialization was slightly more effective with hydroxyectoine than with ectoine. While serum promotes the closure of the gap in general, the positive results achievable through the addition of ectoine/hydroxyectoine were observed especially in the serum-free cultures.

The mean value of the gap widths after 24 h of the sample not treated with ectoine was determined to be 100%. The following gap width percentages could be detected:

| c(Ectoine)/mM | Gap width in % |
|---|---|
| 0 | 100 |
| 0.1 | 100.687 |
| 0.5 | 96.434 |
| 1 | 91.632 |
| 2.5 | 93.012 |
| 5 | 88.418 |

### EXAMPLE 3

### Promoting Wound Healing Through Hydroxyectoine and Glucosylglycerol on the Basis of Porcine Ex-Vivo Skin Punches

An ex-vivo porcine wound healing model was used as it has been described in patent publication DE 103 17 400 B4.

### Samples:

| | Concentration (% w/w) | Osmolarity |
|---|---|---|
| Hydroxyectoine I | 3.84 | 302 |
| Hydroxyectoine hypertonic | 6 | 454 |
| Glucosylglycerol I | 4.76 | 305 |
| Glucosylglycerol hypertonic | 7.55 | 476 |
| PBS hypertonic | | 463 |
| PBS isotonic | | |

From the plicae of washed and sterilized pig ears sample punches 6 mm in diameter were taken. From the center of the punches the epidermis and the upper dermis were removed in an area of 3 mm. Immediately after the models had been generated 5 μl of the test substances were introduced into the wounds, while 5 μl of PBS were applied into the control models. After 48 h (t₄₈ₕ) the models were flash-frozen and stored at −80° C.

14 test series each were carried out so that 8 to 11 evaluable models were available for each sample.

Preparation of Sections from the Models:

The models were completely embedded in tissue freezing medium (company of Leica, Nussloch), and 6 μm thin sections were prepared using a cryostat. Care had been taken that the focus was always in the center of the respective model.

The sections were placed on SuperFrost object slides, air dried, fixed for 10 min. in −20° C. cold acetone, and stored at −80° C.

All models were stained with hematoxylin/eosin (2 sections each). The sequence of the staining process was as follows:
1) 6 min. Incubation with hematoxylin
2) briefly rinsing in tap water
3) briefly rinsing in HCl alcohol
4) 15 min. rinsing in running tap water
5) briefly rinsing in distilled water
6) 1 min. Incubation in eosin (0.2%)
7) briefly rinsing in tap water
8) 20 sec. Incubation in distilled water
9) 20 sec. each Incubation in ascending alcohol series (50%, 50%, 96%, 2×100%)
10) 20 sec. Incubation in xylol
11) following this: Drying sections and mounting them with Eukitt

The staining was evaluated by means of a Leica light microscope DM LS and an Olympus Camedia digital camera.

### Statistical Evaluation (Wound Healing Score):

0: No wound healing progress
1: Small wound tongue
2: Large wound tongue
3: Closed sheet
4: Multi-layered closed sheet

In the event that both wound margins could be assessed the average between the left and the right wound margin was determined. The statistical evaluations were made using Student's paired T-test.

### Evaluation:

1) Absolute wound healing progress (all evaluable from 14 models, i.e. without models with 2 hair follicles in the wound or large craters), HE: Hydroxyectoine; GG: Glucosylglycerol; MW: Mean value; σ: Standard deviation:

| | MW | σ | SEM |
|---|---|---|---|
| HE I | 1.92 | 0.96 | 0.30 |
| HE hypertonic | 2.08 | 0.72 | 0.23 |
| GG I | 2.12 | 0.81 | 0.31 |
| GG hypertonic | 1.66 | 0.93 | 0.30 |
| PBS hypertonic | 1.60 | 0.84 | 0.28 |
| PBS isotonic | 1.84 | 0.81 | 0.26 |

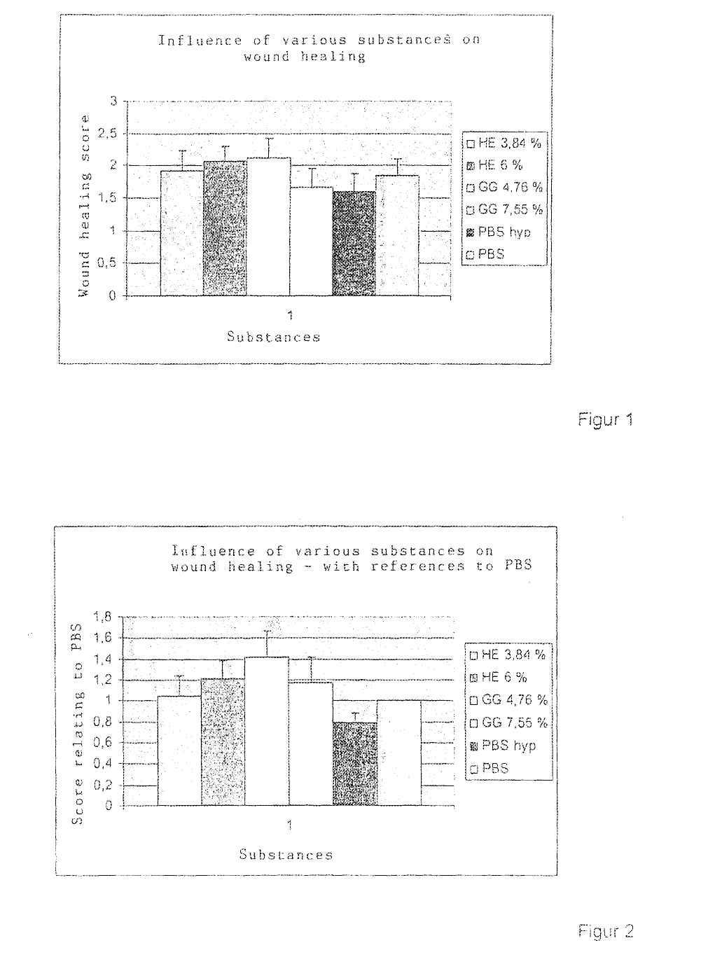

FIG. 1 shows the results in the form of a bar chart. The standard deviations are indicated as dashes above the bars (PBS=PBS isotonic).
2) Wound healing progress based on the PBS control; this evaluation is made to take into account the wound healing potential of the individual pig determined by the values achieved with PBS:

| | MW | σ | SEM |
|---|---|---|---|
| HE I | 1.05 | 0.62 | 0.20 |
| HE hypertonic | 1.21 | 0.53 | 0.17 |
| GG I | 1.42 | 0.67 | 0.25 |
| GG hypertonic | 1.17 | 0.78 | 0.25 |
| PBS hypertonic | 0.78 | 0.30 | 0.10 |
| PBS isotonic | 1.00 | 0 | 0 |

FIG. 2 shows the results in the form of a bar chart. The standard deviations are indicated as dashes above the bars (PBS=PBS isotonic).

Both hydroxyectoine and glucosylglycerol were found to offer improved wound healing potential.

1. Composition containing as active agent ectoine, hydroxyectoine, glucosylglycerol and/or salts, esters or amides of these compounds for promoting the regeneration of injured body tissue.

2. Composition according to claim 1, characterized in that the body tissue is skin or mucous membrane.

3. Composition according to claim 1, characterized in that the injury of the body tissue is of traumatic nature.

4. Composition according to claim 1, characterized in that the injury of the body tissue is an ulcer.

5. Composition according to claim 1, characterized in that the injury is a chronic injury, in particular a chronic wound or a chronic ulcer.

6. Composition according to claim 5, characterized in that the injury of the body tissue is due to the diabetic foot syndrome.

7. Composition according to claim 1, characterized in that the injury of the body tissue is due to a decubitus ulcer.

8. Composition according to claim 1, characterized in that the injury of the body tissue is an anal fissure or hemorrhoids injury.

9. Composition according to claim 1, characterized in that the glucosylglycerol is 2-O-α-glucosylglycerol or 2-O-β-glucosylglycerol.

10. Composition according to claim 9, characterized in that the glucosylglycerol is 2-O-α-D-glucosylglycerol.

11. Composition according to claim 1, characterized in that the composition contains a substance or a plurality of further substances to be selected from: dexpanthenol or derivatives, arnica montana extract (arnica), capsaicin, capsicum extract, hypericum perforatum extract (St John's wort), cardiospermum halicacabum (balloon plant), hamamelis virginiana extract (witch hazel), tocopherol, allantoin, bisabolol, cocoa extract, silver, nanosilver, microsilver, amorphous silver, salts of silver, zinc, zinc oxide, calendula officinalis extract (marigold), honey and honey extracts, propolis, melilotus officinalis extract, comfrey extract (symphytum), echium vulgare extract, cumin, angelica sinensis extract, ferulic acid, hyaluronic acid, aloe vera extract, matricaria recutita (chamomile) extract, allium cepa (onion) bulb extract, achillea millefolium extract (yarrow), glycyrrhiza inflate extract (licorice), licochalcon A, silicone, urea, echinacea purpurea (purple coneflower) extract, chicoric acid.
